(19) United States
(12) Patent Application Publication
(10) Pub. No.: US 2023/0405330 A1
(43) Pub. Date: Dec. 21, 2023

(54) SYSTEMS AND METHODS FOR STIMULATION PARAMETER CONTRAST (SPC) IMAGING

(71) Applicant: Case Western Reserve University, Cleveland, OH (US)

(72) Inventor: Michael Moffitt, Solon, OH (US)

(21) Appl. No.: 18/337,677

(22) Filed: Jun. 20, 2023

Related U.S. Application Data
(60) Provisional application No. 63/353,722, filed on Jun. 20, 2022.

(51) Int. Cl. A61N 1/36; A61N 1/05

(52) U.S. Cl. CPC A61N 1/36139; A61N 1/0534

(57) ABSTRACT

An improved neural mapping technique can use two stimulations. The first stimulation can be used to excite a first group of neural elements with a first stimulation parameter set. After the first group of neural elements has entered a refractory state, a second group of neural elements can be excited with a second stimulation parameter set. The response to at least the second stimulation parameter set can be measured and at least one property of constituents of the first group of neural elements and at least one property of constituents of the second group of neural elements can be estimated.

### CROSS-REFERENCE TO RELATED APPLICATIONS

This application claims the benefit of U.S. Provisional Application Ser. No. 63/353,722, filed 20 Jun. 2022, entitled “SYSTEMS AND METHODS FOR STIMULATION PARAMETER CONTRAST (SPC) IMAGING”. The entirety of the provisional application is incorporated by reference for all purposes.

### TECHNICAL FIELD

The present disclosure relates generally to deep brain stimulation (DBS) and, more specifically, to systems and methods to study stimulus-evoked field potentials using stimulation parameter contrast (SPC) imaging.

### BACKGROUND

Neurological disorders can cause a significant number of disabilities and deaths each year. Current treatments for neurological disorders are at best insufficient. Pharmaceuticals are the most widely accepted treatment modality, but the systemic nature of pharmaceuticals often leads to undesirable side effects. Accordingly, dose constraints are applied when pharmaceuticals are used in order to attempt to control these undesirable side effects. Such dose constraints can hamper the effectiveness of pharmaceutical treatments. Additionally, many neurological disorders do not respond to treatment with pharmaceuticals and are referred to as pharmaceutical refractory disorders. Implantable electrical stimulation, such as deep brain stimulation (DBS), represents a complimentary and/or stand-alone tool to treat neurological disorders through a localized excitation of neural elements via surgically implanted electrodes. However, DBS has not been widely adopted into clinical practice, potentially due to a lack of mechanistic understanding, a lack of biomarkers and related tools to address practical clinical bottlenecks, and/or a variability in outcomes for different patients. Thus, treatment options for neurological disorders are currently severely limited by lack of research in the implantable electrical stimulation space.

### SUMMARY

Improved mapping and biomarker techniques may be used to investigate mechanisms of action and increase mechanistic understanding of implantable electrical stimulation, address clinical bottlenecks (e.g., shortening surgical time and simplifying device programming), and/or reduce variability in outcomes across patients. One such improved mapping and biomarker technique is stimulation parameter contrast (SPC) imaging that can be used to study stimulus-evoked field potentials. With SPC imaging, high-resolution activation maps can be generated based on the stimulus-evoked field potentials of neural elements.

In an aspect, the present disclosure can include a system that can be used for SPC imaging. The system can include at least one implantable electrode configured to apply electrical stimulation; and a computing device, coupled to the at least one implantable electrode. The computing device includes a memory storing instructions and a processor to execute the instructions to at least: configure a first stimulation comprising a first parameter set to be delivered to a location in a stimulation neighborhood to excite a first group of neural elements at the location in the stimulation neighborhood; after the first stimulation is applied, collect data related to an excitation of the first group of neural elements; configure a second stimulation comprising a second parameter set to be delivered to the location in the stimulation neighborhood after the first group of neural elements has entered a refractory state from the first stimulation to excite a second group of neural elements at the location in the stimulation neighborhood; after the second stimulation is applied, collect data related to an excitation of the second group of neural elements; and estimate properties of constituents of the first group of neural elements and properties of constituents of the second group of neural elements based on the data related to the excitation of the first group of neurons and the data related to the excitation of the second group of neurons.

In another aspect, the present disclosure can include a method for SPC imaging. The method can include: configuring, by a system comprising a processor, a first stimulation comprising a first parameter set to be delivered to a first location in a stimulation neighborhood to excite a first group of neural elements at the location in the stimulation neighborhood; after the first stimulation is applied, collecting, by the system, data related to an excitation of the first group of neural elements; configuring, by the system, a second stimulation comprising a second parameter set to be delivered to a second location in the stimulation neighborhood after the first group of neural elements has entered a refractory state from the first stimulation to excite a second group of neural elements at the second location in the stimulation neighborhood; after the second stimulation is applied, collecting, by the system, data related to an excitation of the second group of neural elements; and estimating, by the system, at least one property of constituents of the first group of neural elements and at least one property of constituents of the second group of neural elements based on the data related to the excitation of the first group of neurons and the data related to the excitation of the second group of neurons.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing an example of a system that can be used for stimulation parameter contrast (SPC) imaging in accordance with an aspect of the present disclosure;

FIG. 2 is a diagram showing an example of the Stim Contrast Core module that can be executed by the system of FIG. 1;

FIG. 3 is a process flow diagram illustrating a method for estimating neural properties in accordance with another aspect of the present disclosure;

FIG. 4 is a process flow diagram illustrating a method for performing SPC imaging in accordance with another aspect of the present disclosure;

FIG. 5 shows an example of the Stim Contrast Core in comparison to a traditional single pulse;

FIG. 6 shows examples of multiple current sources used to repeat the stim, contrast core at several stimulation locations to create a spatial map;

FIG. 7 shows example stimulation patterns used for polarity contrast imaging, a subset of SPC imaging; and

FIG. 8 shows various measurable features that can be used in a given SPC image.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an”, and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “neurological disorder” refers to a disease or abnormality of a portion of a patient's nervous system and can be characterized by different signs and symptoms. A neurological disorder may affect any portion of a patient's nervous system, but in some instances the neurological disorder may affect at least a portion of the brain (e.g., Parkinson's disease, essential tremor, dystonia, dementia, stroke, depression, Obsessive Compulsive Disorder (OCD), epilepsy, tinnitus, Tourette syndrome, schizophrenia, pain, hypertension, obesity, addiction, and the like). Such neurological disorders contribute to large amounts of disability and death globally and have many direct and indirect economic costs.

As used herein, the terms “deep brain stimulation” and “DBS” refer to a complimentary or stand-alone electrical stimulation of an area within the brain, aiming to treat one or more symptoms of neurological disorders that uses one or more implanted electrodes (e.g., electrode arrays, electrode leads, etc.) for very localized excitation of neural elements associated with the symptoms.

As used herein, the terms “stimulation parameter contrast imaging” and “SPC imaging” refers to a technique that uses stimulation response differences of different neural elements to estimate the types of neural elements at a given location in a stimulation neighborhood. The stimulation response differences can be represented in one or more types of conduction maps, as an example.

As used herein, the term “neural element” can refer to any part of the brain or central nervous system that can conduct an electrical stimulation. A neural element can be, for example, neurons, nerve fibers, neural cells, axons of passage, axon terminals, or the like, in the brain. Neural elements can have various sizes, orientations, and electrical stimulation response dynamics. Populations of neural elements can be grouped together based on at least one of location and electrical stimulation response characteristics (e.g., how the neural elements respond to stimulations with different properties (e.g., polarity/field orientation, pulse width, frequency, duty cycle/burst/patterns, amplitude, or the like)).

As used herein, the term “neural environment” can refer to any area that contains one or more types of neural elements. As an example, one or more neural environments can be within a stimulation neighborhood.

As used herein, the term “stimulation neighborhood” can refer to an area along and/or around a stimulation area (e.g., an area around an electrode, a lead, etc.). A stimulation neighborhood can include one or more neural environments and/or types of neural elements.

As used herein, the term “stimulation” can refer to delivery of a signal (e.g., an electrical signal) to activate conduction within at least one neural element. A stimulation signal can have either a cathodic or an anodic polarity. Applying a stimulation to a neuron, or other neural element, can result in the neuron, or other neural element, undergoing an excitation period and a refractory period.

As used herein, the term “excitation period” can refer to the time period when a neural element is in a state of depolarization until an action potential is generated.

As used herein, the term “refractory period” can refer to the period after an action potential is generated in a neural element when the ion channels in the cellular membrane of the neural element reach a state in which a subsequent action potential cannot be generated (absolute refractory period) or require a stronger stimulus (relative refractory period).

As used herein, the term “absolute refractory period” can refer to the period immediately following the firing of a neural element when the neural element cannot be activated, regardless of the strength of the stimulus applied. The absolute refractory period starts immediately after the initiation of the action potential and lasts until after the peak of the action potential.

As used herein, the term “relative refractory period” can refer to the period immediately following the absolute refractory period when partial repolarization has occurred and a greater than normally needed stimulus is needed in order to elicit a second action potential.

As used herein, the terms “subject” and “patient” can be used interchangeably and refer to any warm-blooded organism including, but not limited to, a human being, a pig, a rat, a mouse, a dog, a cat, a goat, a sheep, a horse, a non-human primate, a rabbit, a cow, etc.

### II. Overview

Deep brain stimulation (DBS), an implanted electrical stimulation technique, is effective as a complimentary therapy to pharmaceuticals or as a stand-alone therapy for patients suffering from pharmaceutical refractory movement disorders that are pharmacologically resistant (or other neurological disorders). However, DBS is not as widely used in clinical practice as it could be at least in part because of clinical bottle necks such as the mechanisms by which DBS therapy works not being well understood, the biomarkers and related tools to address practical clinical bottlenecks of DBS being unknown, and not well understood variability in outcomes of patients that undergo DBS. The clinical bottlenecks, for example, may be due to difficulties with surgical placement of the electrodes (e.g., arrays and/or leads) and/or programming a device to configure parameters of the stimulation, both of which are time intensive, difficult to optimize, and lack clear biomarker guidance. Improved mapping and biomarker techniques are needed to facilitate both surgical placement of electrodes and the device programming in order to remove clinical bottle necks for improving the use of DBS based treatment for neurological disorders.

Stimulation Parameter Contrast (SPC) imaging provides just such an improved mapping and biomarker technique. SPC imaging uses the stimulation response differences of distinct neural element classes to estimate the types of neural elements at a given location in a stimulation neighborhood. Using a “Stim Contrast Core” type of stimulation (described herein), a first group of neural elements can be excited with a first stimulation parameter set, then after the first group of neural elements has entered a refractory state (ideally the absolute refractory period) a second group of neural elements can be excited with a second stimulation parameter set and the response to the second parameter set can be measured. The Stim Contrast Core reveals the response of the specific neural elements that are not excitable by the first stimulation parameter set but are excitable by the second stimulation parameter set. In fact, stimulations can be configured with the Stim Contrast Core and can be repeated with different stimulation parameters and/or at several stimulation locations to create a spatial map of the neural elements and their responses. Before repeating, enough time is permitted to lapse to allow the neural elements to return to a baseline. Using the results of the Stim Contrast Core, stimulus-evoked field potential maps can be created to model and develop clinically useful neuroimaging (e.g., conduction/activation maps) and electrophysiology biomarkers.

### III. Systems

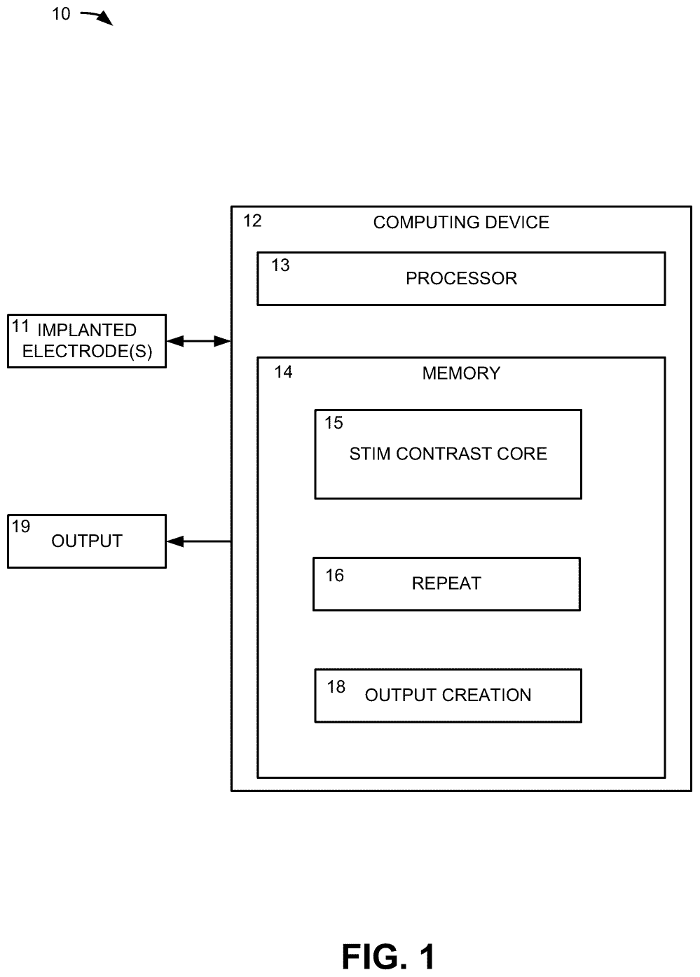

An aspect of the present disclosure relates to a system 10 (shown in FIG. 1) to study stimulus-evoked field potentials in neural elements using stimulation parameter contrast (SPC) imaging. Neural elements can include cells, axons of passage, axon terminals, or the like and can have various sizes, orientations, electrical stimulation response dynamics, and the like. Any given neural environment can include one or more different types of neural elements. Neural elements with different characteristic (e.g., different types of neural elements) can respond differently (or do not respond at all) to electrical stimulation. Groups of neural elements may respond differently depending on the stimulation properties applied at a given time. Stimulation properties that can be varied can include, polarity/field orientation, pulse width, frequency, duty-cycle/burst patterns, or the like.

SPC imaging creates neural maps by using measured stimulation response differences of distinct neural element classes to estimate the types of neural elements at given locations in a stimulation neighborhood (e.g., along and/or around each lead used to apply stimulation and measure responses). To do so, SPC imaging uses a module call the Stim Contrast Core to reveal the response of specific neural elements. The Stim Contrast Core module can, at a base (1) excite a first group of neural elements by applying a first electrical stimulation with a first stimulation parameter set (e.g., cathodic stimulation) at a location, (2) wait for the first group of neural elements to enter a refractory state (ideally the absolute refractory period) (this can be predetermined with test stimulations), (3) excite a second group of neural elements by applying a second electrical stimulation with a second stimulation parameter set (e.g., anodic stimulation) at the location, and (4) measure (e.g., using additional electrode contact(s) on the lead) the response to the second stimulation pattern. The Stim Contrast Core reveals the response of the specific neural elements that are not excitable by the first stimulation parameter set but are excitable by the second stimulation parameter set. This can be repeated for any number of stimulation parameter sets and stimulation locations to create spatial maps of the responses of each neural element types (in locations surrounding the lead) to various stimulation parameters. Images of the spatial maps can be provided to illustrate the contrast within the stimulus-evoked field potentials.

The system 10 includes at least one implantable electrode (represented as implanted electrode(s) 11) configured to apply an electrical stimulation to at least one location in a brain. For example, the at least one implantable electrode can be in a DBS lead, which may include two or more implantable electrodes. A stimulation area can be defined based on the location of the at least one implantable electrode (e.g., at least a portion of a stimulation neighborhood). The stimulation neighborhood can include one or more different neural elements. Neural elements can be within the stimulation neighborhood and/or proximal to the stimulation neighborhood but in electrical communication with the stimulation neighborhood).

The system 10 also includes a computing device 12, that can be coupled to the at least one implantable electrode (e.g., by a wired connection and/or a wireless connection). The computing device 12 can include a memory 14 storing instructions and a processor 13 configured to access the memory to execute the instructions. The instructions can be included in/embodied by one or more modules, such as the Stim Contrast Core module 15, the Repeat module 16, or the Output Creation module 18.

As an example, the executable instruction stored in the memory 14 and executed by the processor 13 as part of the Stim Contrast Core module 15 can include the following. Configure a first stimulation, which includes a first parameter set, to be delivered (e.g., by at least one of the implanted electrodes 11) to a location in a stimulation neighborhood to excite a first group of neural elements at the location in the stimulation neighborhood or in electrical communication with the location (e.g., are affected by a stimulation at the location). After the first stimulation has been applied, collect data related to an excitation of the first group of neural elements. The data can be, for example, conduction and/or timing data related to the first group of neural elements. Then configure a second stimulation, which includes a second parameter set, to be delivered to the location in the stimulation neighborhood (e.g., by at least one of the implanted electrodes 11) after the first group of neural elements has entered a refractory state from the first stimulation to excite a second group of neural elements at the location in the stimulation neighborhood or in electrical communication with the location. The first parameter set and the second parameter set can each include at least one different parameter, for example, pulse widths, amplitudes, burst properties, field shape/orientation/polarity, frequency, duty-cycle, pulse shape, or the like. After the second stimulation is applied, collect data related to an excitation of the second group of neural elements, and estimate properties of constituents of the first group of neural elements and/or properties of constituents of the second group of neural elements (the properties can be related to conduction, for example) based on the data related to the excitation of the first group of neurons and the data related to the excitation of the second group of neurons. These instructions can be repeated 16 with different stimulation parameter sets and/or different locations of stimulation. Additionally, the processor 13 can perform tasks, such as defining the location extending for a distance around the at least one implantable electrode 11, automatically providing information about the location, and modifying the first stimulation and/or the second stimulation based on the data related to the excitation of the first group of neurons and/or the data related to the excitation of the second group of neurons. The data can be collected by the at least one implanted electrode 11 (e.g., the same electrode through which a stimulus is delivered, a separate electrode on the same lead, a separate electrode on another lead, an electrode configured for measuring electrical responses, or the like), an imaging device coupled to the computing device, or another sensor (e.g., implanted electrode, surface electrode, or the like) (not shown) coupled to the computing device 12.

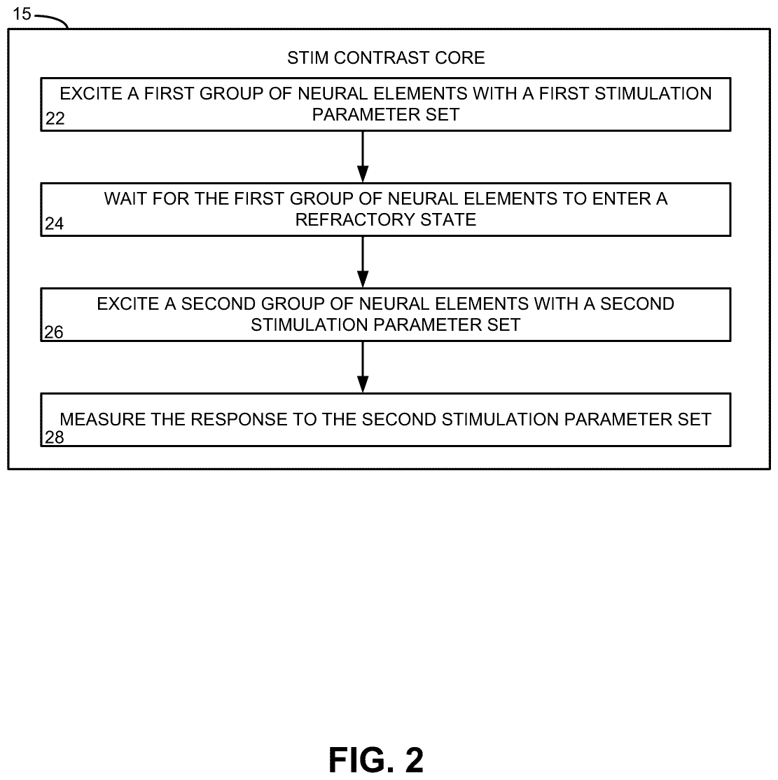

Described more broadly, the processor 13 can execute the Stim Contrast Core module 15, the Repeat module 16, and then the output creation module 18. Shown in FIG. 2 is an example of the Stim Contrast Core module 15 that can be defined and executed by the system 10. Simply, the Stim Contrast Core module 15 involves exciting a first group of neural elements with a first stimulation parameter set 22, waiting for the first group of neural elements to enter a refractory state 24 (e.g., the absolute refractory state and/or the relative refractory state, depending on the instance), exciting a second group of neural elements with a second stimulation parameter set 26, and measuring the response to the second parameter set 28. The Stim Contrast Core module 15 can be repeated, by Repeat module 16 (shown in FIG. 1) after a time allowing the first and second groups of neural elements to recover from the refractory state and re-enter a baseline neural conduction state. The repeating can include the same parameter sets at the same location(s) (e.g., for averaging purposes), different parameter sets at the same location(s) (e.g., for determining what those neural elements respond to and how they respond), the same parameter sets at different location(s) (e.g., to create a larger spatial understanding of the neural elements), and/or different parameter sets at different location(s). The Output Creation module 18 can create an output (e.g., communicated via output 19 in FIG. 1), such as a conduction map, based on the data and estimates determined through the repeated use of the Stim Contrast Core module 15.

Referring again to FIG. 1, the system 10, in some instances, can also include an output 19 device coupled to the computing device 12 by a wired connection and/or a wireless connection. The output 19 device can be, for example, a visual display, an audio output, a printer, and/or the like. The output 19 device can display the conduction maps prepared by the computing device 12. The conduction map can be a spatial map based on estimates of different types of neural elements, a boundary of a neural structure in the stimulation neighborhood, or the like. The conduction map is improved compared to previous conduction maps because the SPC imaging removes some, or all, artifacts from the data collected from the implanted electrode(s) 11 and isolates and measures activation of neural elements responsive to different parameter sets (e.g., different polarities, frequencies, magnitudes, and/or patterns). SPC imaging can characterize neural tissue at given stimulation loci by the neural tissue's anodic and cathodic stimulation response properties with a high resolution. The computing device can output a conduction map that can directly contrast how anodic and cathodic stimulation, for example, interact with local cells and dendrites, axons terminals, and axons of passage in a given area; and/or how anodic versus cathodic stimulation interact with specific neural elements in a variety of vertical and radial orientations. It should be understood, however, that additional differences other than anodic vs. cathodic stimulation can be explored.

### IV. Methods

Another aspect of the present disclosure can include methods for studying stimulus-evoked field potentials using stimulation parameter contrast (SPC) imaging. The methods can be executed using the system shown in FIGS. 1-2, for example. In its simplest form, the system can include a computing device (e.g., computing device 12) to set up a Stim Contrast Core stimulation, receive stimulation data, and create an output based on the stimulation data (e.g., providing information about the stimulus-evoked field potentials) and at least one electrode configured for stimulation and at least one electrode configured for measurement (may be the same electrode). For purposes of simplicity, the methods are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods, nor is the methods necessarily limited to the illustrated aspects.

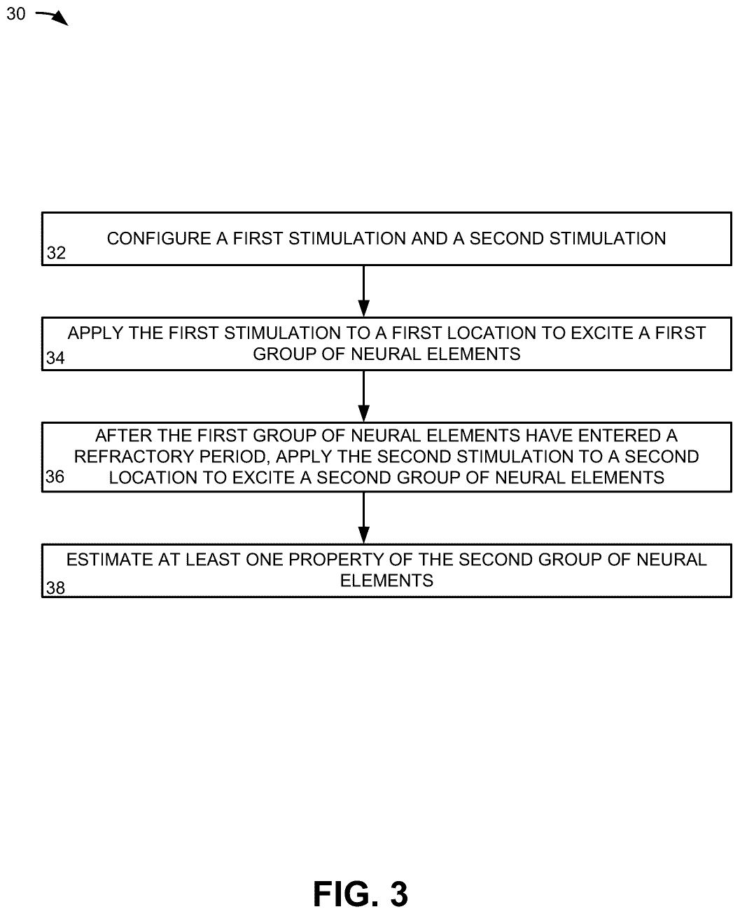

Referring now to FIG. 3, illustrated is a method 30 for estimating neural properties of at least one type of neural elements (generally referred to as the Stim Contrast Core). It should be noted that although two stimulations are described, the method 30 can include more than two stimuli (e.g., 4, 6, 8, etc.). In some instances, an amount of time can pass between sets of two stimuli for neural elements to return to a baseline neural activity level.

At 32, a first stimulation and a second stimulation can be configured with at least one differing parameter (e.g., the first stimulation and the second stimulation can be electrical waveforms, such as a series of one or more pulses). As an example, the first stimulation can be a single pulse, while the second stimulation can be a single pulse or a pulse doublet. The first stimulation can be configured with a first parameter set and the second stimulation can be configured with a second parameter set (with at least one value of at least one parameter of the second set different from at least one other value of the at least one parameter of the first parameter set). The at least one parameter of the parameter sets that can be different can be, for example, pulse widths, amplitudes, burst properties, field shape/orientation/polarity, frequency, duty-cycle, pulse shape, or the like. For example, the first parameter set can establish a cathodic or anodic polarity for the first stimulation and the second parameter set can establish the opposite polarity for the second stimulation.

At 34, the first stimulation can be applied to a first location in a stimulation neighborhood to excite a first group of neural elements. For example, the location can be the location of one or more electrode contacts on a DBS lead, and the one or more electrode contacts can deliver the first stimulation having the first parameter set. In some instances, data related to the excitation of the first group of neural elements can be collected (e.g., by a same or different one or more electrode contacts on the DBS lead). At 36, after the first group of neural elements have entered a refractory period, the second stimulation can be applied to a second location in the stimulation neighborhood (which can be the same as or proximal to the first location) to excite the second group of neural elements. The refractory period can be an absolute refractory period. However, only a relative refractory period is necessary in some cases. The first group of neural elements cannot be excited because they are in a refractory period so only the second group of neural elements can respond. After the second stimulation has been applied, data related to the excitation of the second group of neural elements can be collected (e.g., by the one or more electrodes). The data related to the excitation of the first group of neural elements and the data related to the excitation of the second group of neural elements can be, for example, reflective of different electrical stimulation dynamics responses of various groups of neural elements to different stimulation properties (e.g., polarity, field orientation, pulse width, frequency, duty-cycle/burst/patterns, etc.).

Additionally, it should be noted that the first group of neural elements and the second group of neural elements can be at the same location in a stimulation neighborhood or different locations (remote from one another, adjacent to one another, or overlapping one another) in the stimulation neighborhood (however, in some instances, the locations need not even be in the same stimulation neighborhood). The stimulation neighborhood can be proximal to a deep brain stimulation (DBS) lead that delivers the first stimulation and the second stimulation. The DBS lead can include at least two electrodes, each configured to deliver the first stimulation and/or the second stimulation (and the stimulations can be delivered by the same electrode and/or by different electrodes).

The excitation of the first group of neural elements and the second group of neural elements at another location remote from or distinct but overlapping the location can be recorded. The data related to the excitation of the first group of neural elements and/or the data related to the excitation of the second group of neural elements can be collected in a different neighborhood than the stimulation neighborhood (e.g., due to the stimulation causing an effect upstream or downstream from the stimulation neighborhood).

At 38, at least one property of the second group of neural elements can be estimated based on measured responses of the second group of neural elements. In some instances, at least one property of the first group of neural elements also can be estimated based on measured responses of the first group of neural elements. The property can be related to excitation and/or conduction. For example, the estimation can be based on data related to excitation of the first group of neurons and data related to excitation of the second group of neurons. For example, the estimate of the at least one property of constituents of the first group of neural elements and the at least one property of constituents of the second group of neural elements provide an estimate of different types of neural elements at the location in the stimulation neighborhood. The estimate of different types of neural elements, in some instances, can be further based on known stimulation response differences of distinct neural elements.

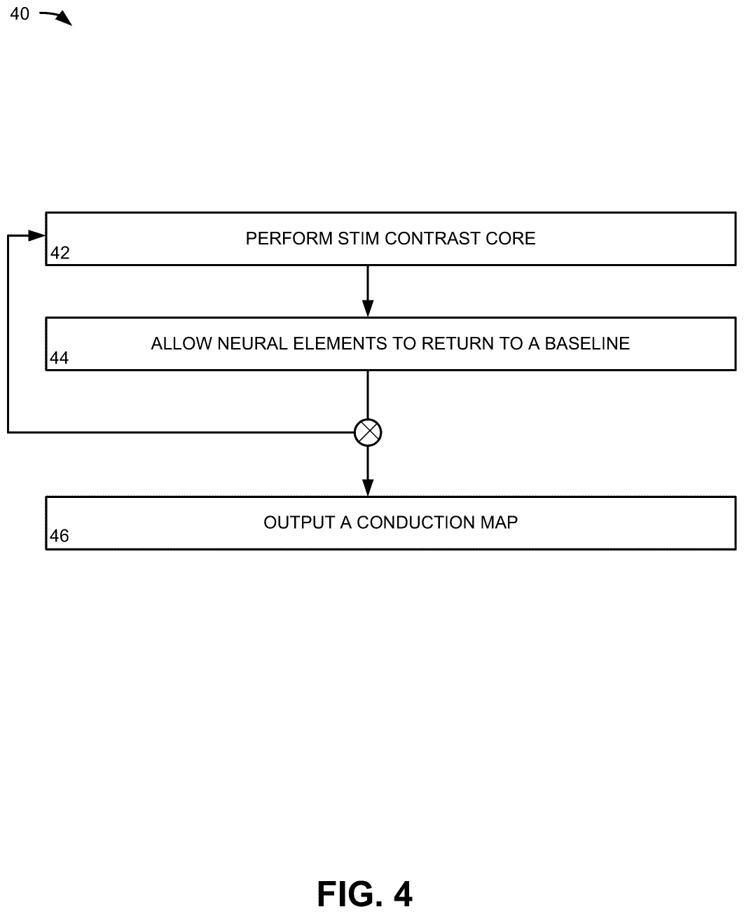

The Stim Contrast Core of the method 30 can be executed/repeated a number of times, as shown in the method 40 of FIG. 4. At 42, the Stim Contrast Core can be performed at a first location and a second location. The first location and the second location can be within the stimulation neighborhood, or the second location can be outside the stimulation neighborhood. The first location and the second location can be the same location or different locations. At 44, the neural elements excited by application of the Stim Contrast Core can be allowed to return to a baseline neural activity level. An amount of time can elapse such that the first group of neural elements and the second group of neural elements return to a baseline neural activity level. Steps 42 and 44 can be repeated such that the Stim Contrast Core can be repeated at the same location(s) or at different location(s), with the same and or/different parameter sets for the first stimulation and the second stimulation. at a second location. For example, steps 42 and 44 can be repeated a number of times necessary to provide data required for a conduction map. At 46, the conduction map can be output based on the one or more applications of the Stim Contrast Core. The conduction map can be a spatial map based on estimates of different types of neural elements, a boundary of a neural structure in the stimulation neighborhood, or the like.

In another example, the method shown in FIG. 3 can also include a third and fourth stimulation. After the first and second groups of neural elements have returned to a baseline neural activity level, then a third stimulation comprising the second parameter set can be delivered to another location in the stimulation neighborhood to excite a third group of neural elements at the location in the stimulation neighborhood. Where the third group of neural elements can include at least one of a portion of the first group of neural elements and a portion of the second group of neural elements, because the stimulation with the second set of parameters could excite the neural elements that were originally stimulated by the stimulation with the first set of parameters but could be stimulated with either set. After the third stimulation is applied, data related to the excitation of the third group of neural elements can be collected. A fourth stimulation comprising the first parameter set can be configured to be delivered to the other location in the stimulation neighborhood, after the third group of neural elements has entered a refractory state from the third stimulation, to excite a fourth group of neural elements at the location in the stimulation neighborhood. The fourth group of neural elements can be a subset of the first group of neural elements. After the fourth stimulation is applied, data related to the excitation of the fourth group of neural elements can be collected. Properties of a response of the first group of neurons and/or the second group of neurons can be estimated based on the data related to the excitation of the third and fourth groups of neural elements, which can include different populations of the first and second groups neurons.

### V. Example Use of SPC Imaging

The systems and methods described above to study stimulus-evoked field potentials using stimulation parameter contrast (SPC) imaging. In some instances, the SPC image or specific points can be taken automatically by a stimulation system (not requiring user initiation at the moment of acquisition). In other instances, the specific points can be compared and processed (and may be recorded as part of a diagnostic report) and the stimulation can be modified (manually or automatically by the computing device) based on the comparison (e.g., SPC may change by medication state, symptom state, etc., and a corresponding change in stimulation may be warranted).

A neural environment includes one or more types of neural elements. Neural elements can be of various sizes/orientations and may exhibit different electrical stimulation dynamics responses to different stimulation properties (e.g., polarity, field orientation, pulse width, frequency, duty-cycle/burst/patterns, etc.). SPC imaging uses the stimulation response differences of distinct types of neural elements at a given location in a stimulation neighborhood using the following construct: excite a first group of neural elements with a first stimulation parameter set; wait for the first group of neural elements to enter a refractory state; excite a second group of neural elements with a second stimulation parameter set; and measure the response to the second stimulation parameter set. The parameter sets can differ, for example, in pulse width, amplitude, burst properties, field shape/orientation/polarity, frequency, duty-cycle, pulse shape, or the like.

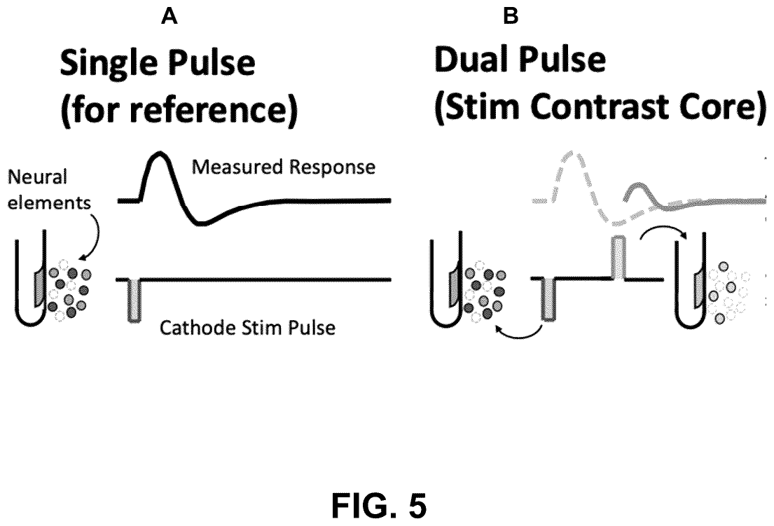

The Stim Contrast Core reveals the response of the specific neural elements that are not excitable by the first stimulation parameter set but are excitable by the second parameter set. FIG. 5 shows an example of the Stim Contrast Core (B) compared to a traditional single pulse (A). The single pulse (A) shows an example of a measured response with a cathode stimulation pulse. The dual pulse Stim Contrast Core (B) has the cathodic stimulation pulse followed by an anodic stimulation pulse. The second anodic stimulation pulse excites distinct neural elements (e.g., distinct from the neural elements excited by the cathodic stimulation pulse) and results in a specific response. In cases where the first and second parameter sets differ by polarity, SPC imaging can be referred to as Polarity Contrast imaging, where estimates of the magnitudes of the contributions of (1) anodic-sensitive, (2) cathodic-sensitive, and (3) non-specific-sensitive neural elements can be estimated and can be shown directly or can be plotted as a ratio or in another mathematically processed form.

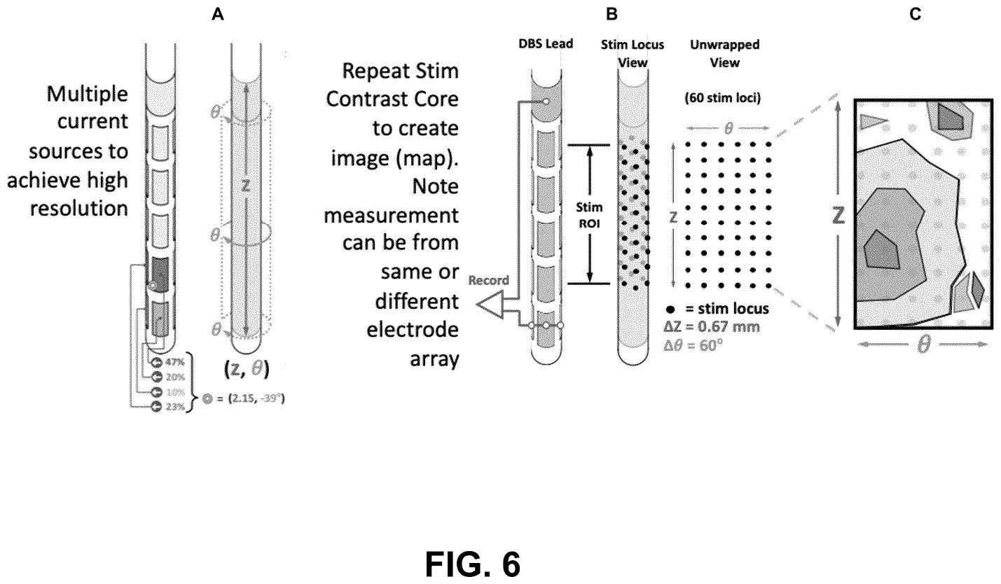

The Stim Contrast Core can be repeated at several stimulation locations to create a spatial map (FIG. 6, elements B and C). Before repeating, in some instances, enough time is permitted to elapse to allow neural elements to return to a baseline. In the spatial activation map in FIG. 6, element C, note that a false color variable can be the response to a specific set of stim parameters or some processed entity (e.g., a ratio of responses of different element types). As an example (FIG. 6, element A) multiple current sources can be used to achieve a high spatial resolution of SPC (e.g., that may even exceed that of the resolution of the physical electrodes).

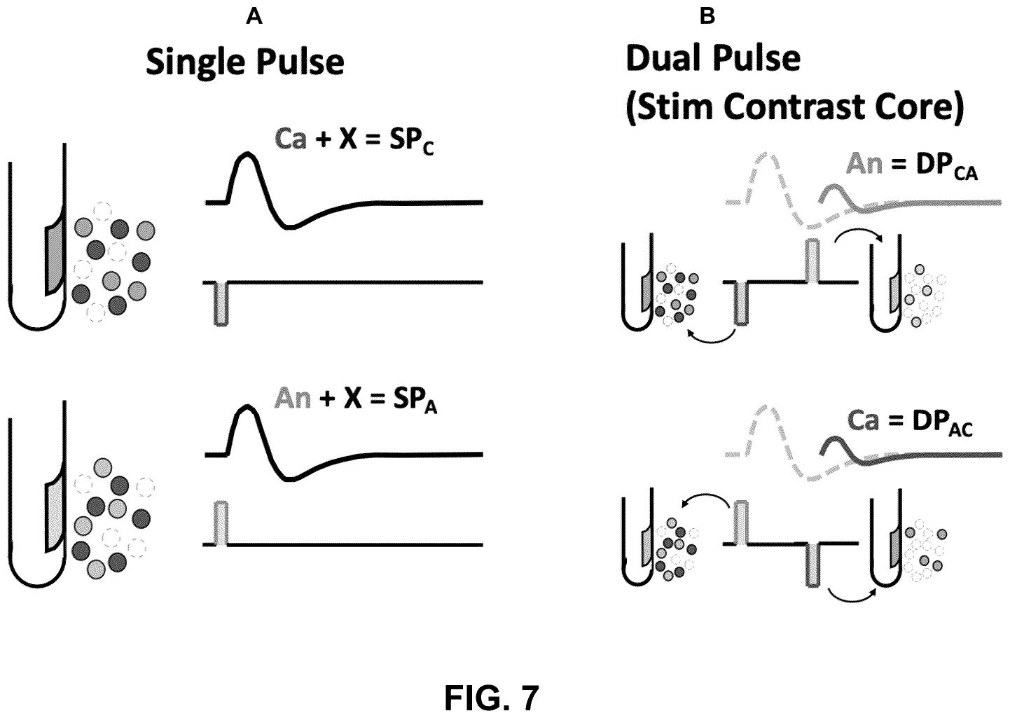

Example stimulation patterns that may be used for polarity contrast imaging, a subset of SPC imaging, are shown in FIG. 7. As shown in elements A and B, SP_{C} is the magnitude of contribution to a specific measurable feature due to Cathodic-sensitive and non-specific-sensitive neural elements, SP_{A} is the magnitude of contribution to a specific measurable feature due to Anode-sensitive and non-specific-sensitive neural elements, Ca=DP_{AC} the magnitude of response due only to Cathodic-sensitive neural elements, and An=DP_{CA} the magnitude of response due only to Anodic-sensitive neural elements. SP_{C}+SP_{A}=Ca+An+2X; (SP_{C}+SP_{A})−(Ca+An)=2X; X=[(SP_{C}+SP_{A})−(Ca+An)]/2, where X is an estimate of the magnitude of contribution to non-specific-sensitive neural elements. The estimated % overlap is 100*X/(X+An+Ca). It should be noted that the estimate is specific to contribution to a given measurable feature (also referred to as a measurement feature). It should be noted that the measurement feature can be recorded in a location at or adjacent to the stimulation location(s), recorded at a location distant to the stimulation location(s), recorded in the basal ganglia of the brain, based on a feature of Evoked Neural Resonant Activity (ERNA), such as ERNA amplitude, frequency, facilitation, or the like, based on a short latency peak (denoted P1, P2, etc. and N1, N2. Etc. for positive and negative, respectively), recorded at the cortex of the brain, the processed output is based on a combination of measurement features, etc.

As an example, the Stim Contrast Core can be repeated at a given location to enable average and variance of responses to be estimated. As another example, the Stim Contrast Core can be repeated by reversing the order (e.g., second stim parameters delivered before first stim parameters) to estimate the response of neural elements sensitive to the first stim parameters. As a further example, three or more sets of stimulation parameters are evaluated during the mapping, where two are selected at a time for execution of the Stim Contrast Core. As another example, a calibration phase can be used to refine stimulation properties before the dual pulse Stim Contrast Core is employed (e.g., the amplitudes of the anodic and cathodic pulses could be different and could be calibrated to yield and equivalent or similar effect size).

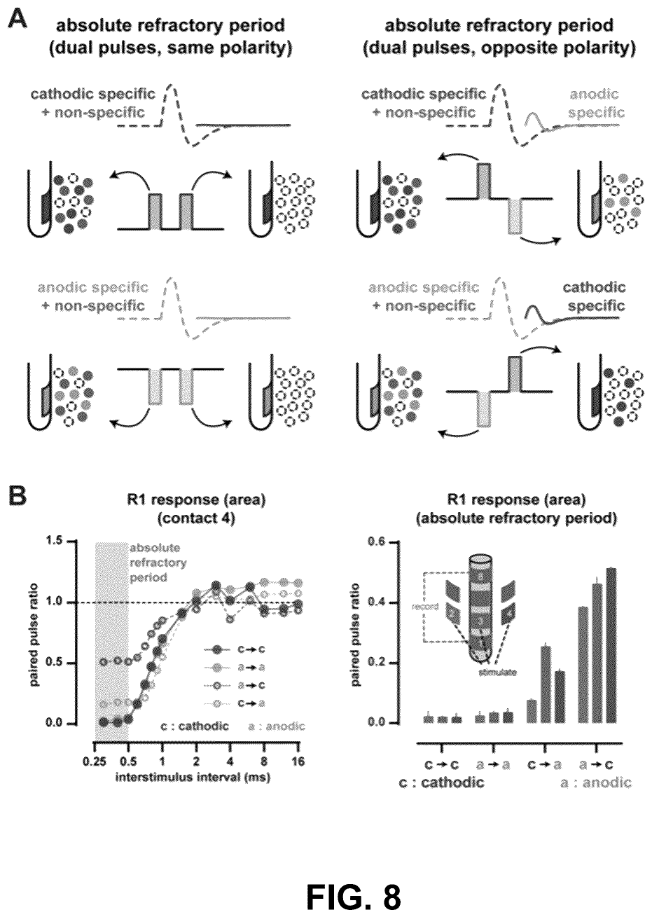

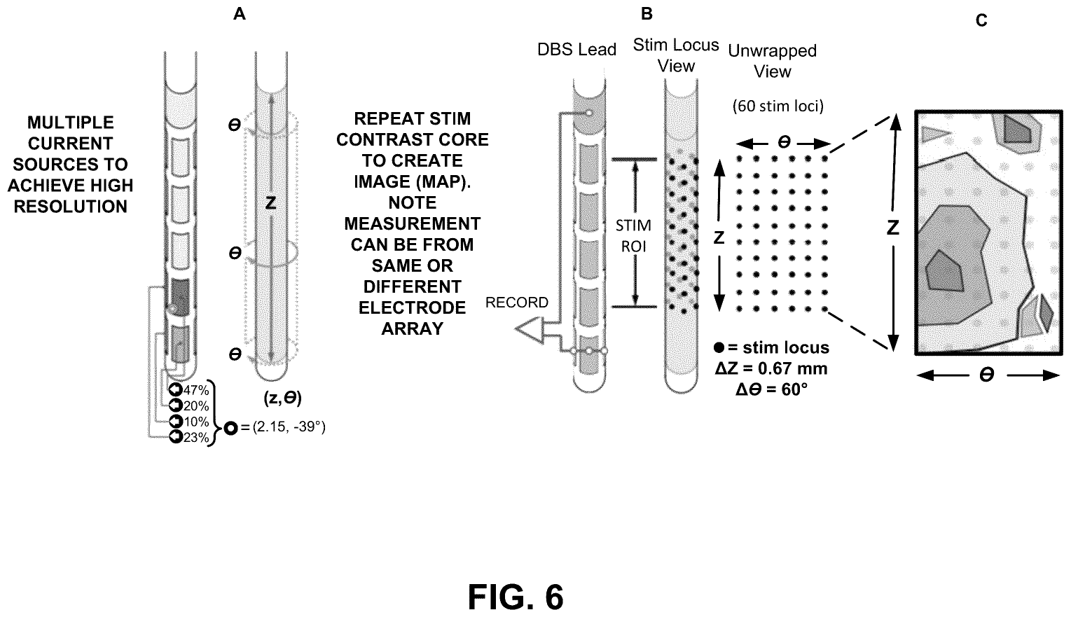

In some instances, the spatial locations and/or properties are distinct between the first set of stimulation parameters and the second set of stimulation parameters to allow for estimation of the amount of overlap between different parameter configurations. For example, the first and second set of stimulation parameters can compare a ring mode to a directional stimulation mode or compare different directional stimulation modes. Ring mode can refer to applying stimulation from a ring of electrode contacts around the DBS lead (e.g., electrode contacts 2, 3, and 4 in FIG. A, element B) all at the same depth. Directional stimulation mode can refer to applying stimulations from one or more electrode contacts facing in a single direction (e.g., left, right, top, bottom, caudal, anterior, posterior, or the like). FIG. 8 shows various measurable features that can be used in a given SP_{C} image (e.g., a comparison between different directional stimulation modes). For example, the measurable feature can be the amount of neural facilitation in a feature that is generated by neural elements sensitive to a given stimulation parameter.

As shown in FIG. 8, element A, dual pulses of the same polarity are shown at the left and dual pulses of opposite polarity are shown at the right. In FIG. 8, element B, anodic neural elements tend to facilitate the R1 measurement at long interstimulus intervals (in other words, not absolute refractory period) (paired pulse ratio >1), while cathodic-sensitive neural elements tend to not affect or depress the R1 measurement of a subsequent pulse (paired pulse ratio <1 or =1). This example shows that a useful method may include a pulse interval that is outside the refractory period. In this case, specificity of neural element type can be traded to see a different effect.

From the above description, those skilled in the art will perceive improvements, changes, and modifications. Such improvements, changes and modifications are within the skill of one in the art and are intended to be covered by the appended claims.

The following is claimed:

1. A method comprising:
configuring, by a system comprising a processor, a first stimulation comprising a first parameter set to be delivered to a first location in a stimulation neighborhood to excite a first group of neural elements at the location in the stimulation neighborhood;
after the first stimulation is applied, collecting, by the system, data related to an excitation of the first group of neural elements;
configuring, by the system, a second stimulation comprising a second parameter set to be delivered to a second location in the stimulation neighborhood after the first group of neural elements has entered a refractory state from the first stimulation to excite a second group of neural elements at the second location in the stimulation neighborhood;
after the second stimulation is applied, collecting, by the system, data related to an excitation of the second group of neural elements; and
estimating, by the system, at least one property of constituents of the first group of neural elements and at least one property of constituents of the second group of neural elements based on the data related to the excitation of the first group of neurons and the data related to the excitation of the second group of neurons.

2. The method of claim 1, wherein the first location and the second location are the same location.

3. The method of claim 1, wherein the first stimulation comprises a single pulse.

4. The method of claim 1, wherein the second stimulation comprises a single pulse or a pulse doublet.

5. The method of claim 1, wherein the stimulation neighborhood is proximal to a deep brain stimulation (DBS) lead that delivers the first stimulation and the second stimulation.

6. The method of claim 5, wherein the DBS lead comprises at least two electrodes, each configured to deliver at least one of the first stimulation and the second stimulation.

7. The method of claim 1, wherein the estimate of the at least one property of constituents of the first group of neural elements and the at least one property of constituents of the second group of neural elements provide an estimate of different types of neural elements in the stimulation neighborhood.

8. The method of claim 7, wherein the estimate of different types of neural elements is further based on known stimulation response differences of distinct neural elements.

9. The method of claim 1, further comprising repeating, by the system, each of the steps at another location in the stimulation neighborhood.

10. The method of claim 9, further comprising creating, by the system, a spatial map based on the estimates of different types of neural elements.

11. The method of claim 9, further comprising estimating, by the system, at least one boundary of a neural structure in the stimulation neighborhood.

12. The method of claim 9, wherein before the repeating, an amount of time elapses so that the first group of neural elements and the second group of neural elements return to a baseline neural activity level.

13. The method of claim 1, wherein the first stimulation and the second stimulation differ in at least one of a polarity, a field orientation, a pulse width, a frequency, a duty-cycle, an amplitude, a burst property, or a pulse shape due to at least one difference between the first parameter set and the second parameter set.

14. The method of claim 1, wherein the first parameter set establishes a cathodic or anodic polarity for the first stimulation and the second parameter set establishes the opposite polarity for the second stimulation.

15. The method of claim 1, wherein the refractory state occurs during an absolute refractory period.

16. The method of claim 1, further comprising recording the excitation of the first group of neural elements and the second group of neural elements at another location in the stimulation neighborhood remote from or distinct but overlapping the first location and/or the second location.

17. The method of claim 1, further comprising:
configuring, by the system, a third stimulation comprising the second parameter set to be delivered to the stimulation neighborhood to excite a third group of neural elements in the stimulation neighborhood;
after the third stimulation is applied, collecting, by the system, data related to an excitation of the third group of neural elements;
configuring, by the system, a fourth stimulation comprising the first parameter set to be delivered to the stimulation neighborhood after the third group of neural elements has entered a refractory state from the third stimulation to excite a fourth group of neural elements at the stimulation neighborhood;
after the fourth stimulation is applied, collecting, by the system, data related to an excitation of the fourth group of neural elements; and
estimating properties of a response of the first group of neurons and/or the second group of neurons based on the data related to the excitation of the third and fourth groups of neural elements.

18. The method of claim 17, wherein the first and second groups of neural elements have returned to a baseline neural activity level before the third stimulation is delivered.

19. The method of claim 1, wherein the data related to an excitation of the first group of neural elements and/or the data related to an excitation of the second group of neural elements is collected in a different neighborhood than the stimulation neighborhood.

20. A system comprising:
at least one implantable electrode configured to apply electrical stimulation;
a computing device, coupled to the at least one implantable electrode, comprising a memory storing instructions and a processor to execute the instructions to at least:
configure a first stimulation comprising a first parameter set to be delivered to a location in a stimulation neighborhood to excite a first group of neural elements at the location in the stimulation neighborhood;
after the first stimulation is applied, collect data related to an excitation of the first group of neural elements;
configure a second stimulation comprising a second parameter set to be delivered to the location in the stimulation neighborhood after the first group of neural elements has entered a refractory state from the first stimulation to excite a second group of neural elements at the location in the stimulation neighborhood;
after the second stimulation is applied, collect data related to an excitation of the second group of neural elements; and
estimate properties of constituents of the first group of neural elements and properties of constituents of the second group of neural elements based on the data related to the excitation of the first group of neurons and the data related to the excitation of the second group of neurons.

21. The system of claim 20, wherein the processor executes the instructions to define the location, wherein the location extends for a distance around the electrode.

22. The system of claim 20, wherein the processor executes the instructions to provide information about the location automatically.

23. The system of claim 20, wherein the processor executes the instructions to modify the first stimulation and/or the second stimulation based on the data related to the excitation of the first group of neurons and/or the data related to the excitation of the second group of neurons.

24. The system of claim 20, wherein the implantable electrode is implantable proximal to the stimulation neighborhood and comprises a deep brain stimulation (DBS) lead that delivers the first stimulation and the second stimulation.

25. The system of claim 24, wherein the DBS lead comprises at least two electrodes, each configured to deliver at least one of the first stimulation and the second stimulation.
